(19) United States
(12) Patent Application Publication
(10) Pub. No.: US 2006/0200870 A1
(43) Pub. Date: Sep. 7, 2006

(54) Methods of treating muscular dystrophy

(76) Inventor: Brian Tseng, Denver, CO (US)

Correspondence Address: SHERIDAN ROSS PC, 1560 BROADWAY, SUITE 1200, DENVER, CO, 80202, US

(21) Appl. No.: 11/286,730

(22) Filed: Nov. 25, 2005

Related U.S. Application Data
(60) Provisional application No. 60/630,612, filed on Nov. 24, 2004.
(60) Provisional application No. 60/695,196, filed on Jun. 28, 2005.

(51) Int. Cl. A01K 67/027

(52) U.S. Cl. 800/14

(57) ABSTRACT

The invention provides methods of preventing, treating and ameliorating symptoms of muscular dystrophy. The invention also provides experimental systems for identifying and evaluating such treatments.

### CROSS REFERENCE TO RELATED APPLICATION

This application claims priority under 35 U.S.C. § 119(e) to U.S. Provisional Patent Application No. 60/630,612 filed Nov. 24, 2004, and to U.S. Provisional Patent Application No. 60/695,196 filed Jun. 28, 2005, which are incorporated herein in their entirety by this reference.

### GOVERNMENT INTEREST

This invention was made with government support under grant NINDS 5R01 NS040109-04 awarded by the National Institutes of Health (NIH). The government has certain rights in the invention.

### FIELD OF THE INVENTION

The invention relates to methods of identifying treatments for, and for treating, muscular dystrophies. The invention provides transgenic animals, as well as to compositions and methods relating to the characterization of gene functions in causing, modifying or overcoming muscular dystrophies. More specifically, the invention provides methods of treating Duchenne Muscular Dystrophy (DMD) through the targeting of creatine and the manipulation of the expression with drug analogs of guanidinoacetate methyltransferase (GAMT) itself or its positive regulators and methods of identifying useful methods of treating DMD in humans.

### BACKGROUND OF THE INVENTION

Duchenne Muscular Dystrophy (DMD) is the most common and severe progressive muscular dystrophy affecting children. It is also the most common inherited lethal defect worldwide, affecting approximately 1 in every 3,500 liveborn males. This pediatric disorder exhibits an inheritance pattern that is X-linked recessive so boys are afflicted whereas girls tend to be carriers. The natural clinical history for boys with DMD is difficulty walking by 3 years of age, wheel-chair bound by 12 years of age, and usually death succumbing to cardio-respiratory failure by 20 years of age. One third of boys afflicted with DMD have no family history but are thought to be spontaneous mutations in the dystrophin gene.

There is no specific therapy for DMD except supportive treatments such as leg braces, surgical tendon releases, and respiratory assistive devices. Pharmacologic treatment includes prednisone 0.75 mg/kg in intermittent regimens which in some patients can help prolong independent ambulation but has significant adverse side-effects including weight-gain, osteopenia, hirsutism, cushingoid facies, behavioral and endocrinology-related issues. Moreover, oral steroids have not been shown to prolong the life expectancy of young men with DMD. Bones develop abnormally, causing skeletal deformities of the chest and other spine. Muscular weakness and skeletal deformities e.g. scoliosis contribute to frequent breathing disorders. Cardiomyopathy occurs in almost all cases. Intellectual impairment is common (30%), but is not inevitable, and does not necessarily worsen as the disorder progresses.

While advances in research have greatly enhanced specific diagnostic tests to identify patients and carriers of DMD, conventional treatments for these patients remain unchanged. To date, there is no specific therapy or treatment for DMD that can extend the expected lifespan of a male afflicted with DMD.

The underlying genetic basis for DMD has been defined as a loss-of-function mutation in the dystrophin gene, which encodes a 427-kD subsarcolemmal protein thought to play crucial roles in linking with transmembrane components and the intracellular cytoskeleton. A mouse model of the DMD disease (mdx) also lacks the same dystrophin protein but, surprisingly, the mice are not similarly crippled. In other words, mdx mice can compensate to a remarkable degree in the absence of dystrophin protein, unlike humans with DMD. In stark contrast to dystrophin-deficient human counterparts, dystrophin-deficient mice (mdx model) are reproductively viable, live near full lifespans, and can run up to 8 km/day (vs the 10 km/day typical of wild-type mice). It is known that the mdx mouse has skeletal muscle histology characterized by minimal fibrosis with over 75% of myofibers having central myonuclei. These are two prominent histologic features considered differential to human DMD (which has more fibrosis scarring and fewer <40% central nuclei), and these differences may reflect desirable molecular adaptations, presumably governed by transcriptional activity. Although there are studies being conducted to describe the function(s) of the dystrophin protein, there is a paucity of information describing how the absence of dystrophin protein manifests differentially into a benign phenotype in the mouse model but not in dystrophin-deficient humans.

The study of model organisms with knockouts or related genetic manipulations is a central theme of many efforts to understand inherited diseases in humans. However, it is not always the case that the phenotype produced by the genetic manipulation of the model organism corresponds well to the human phenotype. Diseases for which mouse knockouts failed to produce human-like phenotypes include Tay-Sachs, Fabry disease, Lesch-Nyhan syndrome and DMD. Although such failures preclude the use of the model system to study etiology directly, differences in susceptibility can themselves be used to interrogate the effects of genetic background on disease, potentially illuminating important protective or compensatory factors that could play a role in the development of novel therapy.

Thus, a mouse model of DMD has been interrogated to understand the regulatory and compensatory mechanisms responsible for producing and overcoming the dystrophin-deficient phenotype and to provide a more readily useful test animal for the study of treatments for DMD. Additionally, there is a long-felt need for treatments for DMD that are not merely palliative but address, or at least compensate for, the underlying genetic abnormalities that lead to DMD in human males.

### SUMMARY OF THE INVENTION

The present invention is drawn to methods of treating muscular dystrophies, and particularly Duchenne Muscular Dystrophy (DMD), by the administration of treatments that overcome the lack of dystrophin protein in DMD patients. The treatments may include, for example, the provision of adaptive molecular therapies that compensate for mutations in the dystrophin gene that result in absent, dysfunctional or marginally-functional dystrophin protein in a mammalian host. In contrast to major efforts directed at replacing dystrophin protein, we propose a novel treatment may also include, for example, the administration of agents that produce an increase in the systemic concentrations of guanidinoacetate methyltransferase (GAMT; EC 2.1.1.2) especially in muscle cells and the substrates therefor and products thereof. The treatments may also include, for example, the administration of agents that produce an increase in the systemic concentrations of arginine:glycine amidinotransferate (AGAT; EC 2.1.4.1) especially in muscle cells and the substrates therefor and products thereof. The treatments may also include, for example, the administration of agents that produce an increase in the systemic concentrations and proper membrane localization of the creatine transporter protein (CRTR) and the substrates therefor and products thereof.

Creatine and creatine phosphate play essential roles in the storage and transmission of phosphate-bound energy. Creatine is synthesized mainly in the liver and pancreas by two reactions. The first, catalyzed by AGAT, transfers the amidino group from arginine to glycine, forming ornithine and guanidinoacetate. The second, catalyzed by GAMT, transfers a methyl group from S-adenosylmethionine to guanidinoacetate, forming creatine which is then transported into the blood stream. Creatine is taken up into tissues such as muscle, by an energy-dependent creatine transporter (CRTR). Trials of oral creatine supplementation have shown some benefits in mdx mice but no such clear benefit in humans with DMD.

The guanidinoacetate methyltransferase (GAMT) gene, which encodes a key enzyme of creatine synthesis, is highly up-regulated in skeletal muscle fibers of dystrophin-deficient mdx mice while the same gene is down-regulated in humans. Without intending to be bound by any one theory, it is believed that the absence of normal dystrophin protein leads to a metabolic derangement precipitated by the chronic leakage and intracellular depletion of creatine phosphagen substrate without accompanying profound skeletal muscle fiber necrosis. This metabolic derangement may reflect a continuum of mild involvement in the mdx mouse which upregulates de novo creatine biosynthetic pathways in skeletal muscle cells to compensate, as opposed to DMD males with profound muscle fiber necrosis and are unable or inadequately upregulate creatine biosynthesis. De novo creatine biosynthesis may be a key metabolic compensatory mechanism to maintain cellular function in spite of ongoing creatine kinase (CK) efflux due to the leaky dystrophin-deficient membrane.

Though it is widely assumed that the elevation of serum creatine kinase (CK) is due to muscle fiber necrosis and loss of intracellular contents, the elevated serum CK could also represent membrane leakage and not cell death/necrosis. Anecdotally, newborn baby boys with DMD have extremely high serum CKs (>30,000 IU/L) yet on muscle biopsies show no features of histopathology. It is therefore believed that elevated serum creatine kinase or muscle cell leakage of CK precedes the major insults that lead to skeletal muscle degeneration or muscular dystrophy. Unfortunately, oral creatine supplementation has not been shown to have a significant therapeutic effect in males with DMD. It is believed that a dystrophin protein deficiency results in improper localization of the creatine transporter protein on the skeletal muscle membranes. Under these circumstances, serum creatine cannot be taken up appropriately into muscle cells. It is also believed that the regulatory elements of the GAMT gene of mice are intrinsically different compared to human GAMT gene and thus responds to different nuclear transcription factors and that these differences may be the underlying component to the species difference in phenotype with dystrophin-deficiency seen in the mdx mouse model. Thus, one embodiment of the present invention is cells that have a disruption in a GAMT gene and a dystrophin gene. The cells of the present invention are comprised of any cells capable of undergoing homologous recombination. Preferably, the cells of the present invention are stem cells and more preferably, embryonic stem cells, and most preferably, murine embryonic stem cells. According to one embodiment, the transgenic cells are produced by introducing a targeting construct into a stem cell to produce a homologous recombinant, resulting in a mutation of the GAMT gene. In another embodiment, the cells are derived from transgenic animals. The cells derived from transgenic animals include cells that are isolated or present in a tissue or organ, and any cell lines or any progeny thereof.

Creatine plays a key role in cellular energy metabolism and is found at high concentrations in metabolically active muscle cells. These cells take up creatine from the extra-cellular fluid by a high affinity Na(+)/Cl(−)-dependent creatine transporter (CRTR). Mutations in the CRTR gene lead to severe retardation of speech and mental development, accompanied by the absence of creatine in the brain. Thus, another embodiment of the present invention is a cell that has a disruption in a CRTR gene and, optionally, a dystrophin gene, an AGAT gene or a GAMT gene. The present inventors have shown that the lack of dystrophin protein in mdx mice and likely in human DMD impairs the proper CRTR membrane localization and potential function of creatine uptake. Also included in the present invention are methods of treating a muscular dystrophy by increasing the expression of the CRTR gene, insuring proper membrane localization of CRTR and increasing the activity of the creatine transporter protein on muscle cell membranes. Through the use of gene therapy, it is contemplated to restore the expression of a creatine transporter protein or increase the expression of fully functional creatine transporter proteins on skeletal muscle membranes. These therapies may be administered before or concurrently with the administration of creatine to a patient suffering a muscular dystrophy and synergistic effects could then optimize a therapeutic effect to enhance a clear clinical benefit.

As noted above, AGAT functions in conjunction with GAMT to produce creatine in the liver and pancreas. Treatment with oral creatine is partially successful in overcoming derangements or deficiencies in AGAT activity. In conjunction with GAMT deficiencies, AGAT deficiencies may be responsible or exacerbate disorders of creatine metabolism and storage and thus foster muscular dystrophies. For this reason, the methods of the present invention may include treating a muscular dystrophy by increasing the expression of the AGAT gene or the activity of the AGAT gene product in skeletal muscle. The host may be treated with gene therapy to replace the expression of a defective AGAT enzyme or increase the expression of fully functional AGAT gene. These therapies may be administered concurrently with methods of replacing or modulating the GAMT and/or CRTR genes as well as the administration of creatine to a patient suffering a muscular dystrophy.

Another embodiment of the present invention is a cell that has a disruption in an AGAT gene and, optionally, a dystrophin gene, a CRTR gene or a GAMT gene.

The invention also provides a targeting construct and methods of producing the targeting construct that, when introduced into stem cells, produces a homologous recombinant. In one embodiment, the targeting construct of the present invention comprises first and second polynucleotide sequences that are homologous to the GAMT gene. The targeting construct also comprises a polynucleotide sequence that encodes a selectable marker that is preferably positioned between the two different homologous polynucleotide sequences in the construct. The targeting construct may also comprise other regulatory elements that may enhance homologous recombination.

The present invention further provides non-human transgenic animals and methods of producing such non-human transgenic animals comprising a disruption in the GAMT, CRTR, AGAT and dystrophin genes. The transgenic animals of the present invention include transgenic animals that are heterozygous and homozygous for one or more mutations in these genes. In one aspect, the transgenic animals of the present invention are defective in the function of at least one of these genes. In another aspect, the transgenic animals of the present invention comprise a phenotype associated with having a mutation in at least one of the GAMT, CRTR, AGAT gene and/or dystrophin gene.

The present invention also provides methods of identifying agents capable of affecting a phenotype of a transgenic animal. For example, a putative agent is administered to the transgenic animal and a response of the transgenic animal to the putative agent is measured and compared to the response of a “normal” or wild type mouse, or alternatively compared to a transgenic animal control (without agent administration). The invention further provides agents identified according to such methods. The present invention also provides methods of identifying agents useful as therapeutic agents for treating conditions associated with a disruption of the dystrophin, GAMT, CRTR and/or AGAT genes.

The present invention further provides a method of identifying agents having an effect on GAMT, CRTR and/or AGAT expression or function. The method includes administering an effective amount of the agent to a transgenic animal, preferably a mouse. The method includes measuring a response of the transgenic animal, for example, to the agent, and comparing the response of the transgenic animal to a control animal, which may be, for example, a wild-type animal or alternatively, a transgenic animal control. Compounds that may have an effect on gene expression or function may also be screened against cells in cell-based assays, for example, to identify such compounds. High through-put screens (HTS) with cell based systems that have introduced reporter constructs e,g, luciferase or green fluorescent protein with 5′ or 3′ UTR and regulatory DNA domains of these genes will enable systematic screening and identification of compounds that could function as agonists for upregulating GAMT/AGAT/CRTR expression or finding compounds that de-repress inhibitors of GAMT/AGAT/CRTR.

The invention also provides cell lines comprising nucleic acid sequences of the GAMT, CRTR and/or AGAT genes. Such cell lines may be capable of expressing such sequences by virtue of operable linkage to a promoter functional in the cell line. Preferably, expression of the GAMT, CRTR and/or AGAT gene sequences is under the control of an inducible promoter. Also provided are methods of identifying agents that interact with GAMT, CRTR and/or AGAT genes, comprising the steps of contacting these genes with an agent and detecting a complex between that putative agent and one of the genes. Such complexes can be detected by, for example, measuring expression of an operably linked detectable marker.

The invention also provides cell lines comprising nucleic acid sequences of the GAMT, CRTR and/or AGAT genes operably linked to a single promoter functional in the cell line. Preferably, expression of these gene sequences is under the control of an inducible promoter.

The invention further provides methods of treating diseases or conditions associated with a disruption in the dystrophin, GAMT, CRTR and/or AGAT gene, and more particularly, to a disruption in the expression or function of the GAMT gene. In a preferred embodiment, methods of the present invention involve treating diseases or conditions associated with a disruption in the GAMT gene's expression or function, including administering to a subject in need, a therapeutic agent that effects GAMT expression or function. In accordance with this embodiment, the method comprises administration of a therapeutically-effective amount of a natural, synthetic, semi-synthetic, or recombinant GAMT gene, GAMT gene products or fragments thereof as well as natural, synthetic, semi-synthetic or recombinant analogs of GAMT gene products.

Similarly, methods of the present invention involve treating diseases or conditions associated with a disruption in the CRTR and/or AGAT genes' expression or functions, including administering to a subject in need, a therapeutic agent that effects the expression or function of these genes. In accordance with this embodiment, the method comprises administration of therapeutically-effective amounts of natural, synthetic, semi-synthetic, or recombinant CRTR/AGAT genes, CRTR/AGAT gene products or fragments thereof as well as natural, synthetic, semi-synthetic or recombinant analogs of these gene products.

The present invention further provides methods of treating diseases or conditions associated with disrupted targeted gene expression or function, wherein the methods comprise detecting and replacing, through gene therapy, mutated at least one of a GAMT/CRTR and/or AGAT gene or a regulatory element of at least one of these genes.

### BRIEF DESCRIPTION OF THE DRAWING

FIG. 1 shows 6 week old mice soleus muscles in whole-cross section in which WT and GAMT null mice show normal muscle morphology whereas mdx mouse demonstrates scattered foci of histopathology.

### DETAILED DESCRIPTION OF THE INVENTION

As used herein, the term “muscular dystrophy” refers to any of a group of hereditary diseases characterized by progressive wasting of muscles including, for example, Duchenne Muscular Dystrophy (DMD) as well as Becker Muscular Dystrophy (BMD), a milder allelic variant of DMD that in some cases does not express even truncated dystrophin protein. Additionally, a muscular dystrophy may include dystroglycanopathies, limb-girdle, merosin deficiency, adhalinopathies, Fukuyama muscular dystrophy, glycosylation defects and the like.

As used herein, the term “gene” refers to (a) a gene containing at least one of the DNA sequences disclosed herein; (b) any DNA sequence that encodes the amino acid sequence encoded by the DNA sequences disclosed herein and/or; (c) any DNA sequence that hybridizes to the complement of the coding sequences disclosed herein. Preferably, the term includes coding as well as noncoding regions, and preferably includes all sequences necessary for normal gene expression including promoters, enhancers and other regulatory sequences.

As used herein, the terms “polynucleotide” and “nucleic acid molecule” are used interchangeably to refer to polymeric forms of nucleotides of any length. The polynucleotides may contain deoxyribonucleotides, ribonucleotides and/or their analogs. Nucleotides may have any three-dimensional structure, and may perform any function, known or unknown. The term “polynucleotide” includes single-, double-stranded and triple helical molecules.

As used herein, “oligonucleotide” refers to polynucleotides of between 5 and about 100 nucleotides of single- or double-stranded DNA. Oligonucleotides are also known as oligomers or oligos and may be isolated from genes, or chemically synthesized by methods known in the art. A “primer” refers to an oligonucleotide, usually single-stranded, that provides a 3′-hydroxyl end for the initiation of enzyme-mediated nucleic acid synthesis. The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A nucleic acid molecule may also comprise modified nucleic acid molecules, such as methylated nucleic acid molecules and nucleic acid molecule analogs. Analogs of purines and pyrimidines are known in the art, and include, but are not limited to, aziridinycytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudouracil, 5-pentylnyluracil and 2,6-diaminopurine. The use of uracil as a substitute for thymine in a deoxyribonucleic acid is also considered an analogous form of pyrimidine. A “fragment” of a polynucleotide is a polynucleotide comprised of at least 9 contiguous nucleotides, preferably at least 15 contiguous nucleotides and more preferably at least 45 nucleotides, of coding or non-coding sequences.

As used herein, the term “gene targeting” refers to a type of homologous recombination that occurs when a fragment of genomic DNA is introduced into a mammalian cell and that fragment locates and recombines with endogenous homologous sequences.

As used herein, the term “homologous recombination” refers to the exchange of DNA fragments between two DNA molecules or chromatids at the site of homologous nucleotide sequences.

As used herein, the term “homologous” as used herein denotes a characteristic of a DNA sequence having at least about 70 percent sequence identity as compared to a reference sequence, typically at least about 85 percent sequence identity, preferably at least about 95 percent sequence identity, and more preferably about 98 percent sequence identity, and most preferably about 100 percent sequence identity as compared to a reference sequence. Homology can be determined using a “BLASTN” algorithm. It is understood that homologous sequences can accommodate insertions, deletions and substitutions in the nucleotide sequence. Thus, linear sequences of nucleotides can be essentially identical even if some of the nucleotide residues do not precisely correspond or align. The reference sequence may be a subset of a larger sequence, such as a portion of a gene or flanking sequence, or a repetitive portion of a chromosome.

As used herein, the term “target gene” (alternatively referred to as “target gene sequence” or “target DNA sequence” or “target sequence”) refers to any nucleic acid molecule or polynucleotide of any gene to be modified by homologous recombination. The target sequence includes an intact gene, an exon or intron, a regulatory sequence or any region between genes. The target gene comprises a portion of a particular gene or genetic locus in the individual's genomic DNA. As provided herein, the target gene of the present invention is the GAMT gene. A “GAMT gene” refers to a sequence that is at least 80% homologus to a gene sequence identified in any of Genebank Accession Nos.: AF010499, NM138924, NM000156, and AF015887. The molecular structure of the guanidinoacetate protein has been elucidated through the crystallographic structure published in 2002 (Komoto, J., et al., J. Mol. Biol. 320(2):223-35 (2002)).

As provided herein, another target gene of the present invention is the L-arginine-glycine amidinotransferate (AGAT) gene (alternatively referred to glycine amidinotransferase, GATM). An “AGAT gene” refers to a sequence that is at least 80% homologus to a gene sequence identified in any of Genebank Accession Nos.: 1432573, AAH03879, AAT39893, AK007325.

As provided herein, another target gene of the present invention is the human creatine transporter (CRTR alternatively abbreviated CRT, CrT and CreaT) gene. A “CRTR gene” refers to a sequence that is at least 80% homologus to a gene sequence identified in any of NCBI Accession Nos.: AAH24444, AAH49801, and AAL66354. Other variants due to alternative splicing may also exist as well as other CRTR-like gene (isoforms).

“Disruption” of the GAMT gene occurs when a fragment of genomic DNA locates and recombines with an endogenous homologous sequence. These sequence disruptions or modifications may include insertions, missense, frameshift, deletion, or substitutions, or any combination thereof. Insertions include the insertion of entire genes, which may be of animal, plant, fungal, insect, prokaryotic, or viral origin. Disruption, for example, can alter a GAMT promoter, enhancer, or splice site of the GAMT gene, and can alter the normal gene product by inhibiting its production partially or completely or by enhancing the normal gene product's activity.

The term, “transgenic cell”, refers to a cell containing within its genome a gene such as the GAMT, AGAT or CRTR gene, that has been disrupted, modified, altered completely or partially by the method of gene targeting.

The term “transgenic animal” refers to an animal that contains within its genome a specific gene that has been disrupted by the method of gene targeting. The transgenic animal includes both the heterozygote animal (i.e., one defective allele and one wild-type allele) and the homozygous animal (i.e., two defective alleles). The term “transgenic mouse” or “transgenic mice” refers to a mouse or to mice containing within the genome a specific gene that has been disrupted by the method of gene targeting. The transgenic mouse includes both the heterozygote mouse (i.e., one defective allele and one wild-type allele) and the homozygous mouse (i.e., two defective alleles).

As used herein, the terms “selectable marker” or “positive selection marker” refers to a gene encoding a product that enables only the cells that carry the gene to survive and/or grow under certain conditions. For example, plant and animal cells that express the introduced neomycin resistance (Neo^{r}) gene are resistant to the compound G418. Cells that do not carry the Neo^{r} gene marker are killed by G418. Other positive selection markers are known to those of skill in the art.

As used herein, the term “modulates” refers to the inhibition, reduction, increase or enhancement of the GAMT and or AGAT function, expression, activity, or alternatively a phenotype associated with a disruption in the GAMT and/or AGAT genes.

As used herein, the term “ameliorates” refers to a decreasing, reducing or eliminating of a pathologic condition, disease, disorder, or phenotype, including an abnormality or symptom associated with a disruption in the GAMT and/or ATAT genes.

The invention is based, in part, on the evaluation of the expression and role of genes and gene expression products, primarily those associated with the GAMT, CRTR and/or AGAT genes. Among others, the invention permits the definition of disease pathways and the identification of diagnostically- and therapeutically-useful targets for the treatment of pathologies involving cellular membrane leaking, including muscular dystrophies and particularly DMD. For example, genes that are mutated or down-regulated under disease conditions may be involved in causing or exacerbating the disease condition. Treatments directed at up-regulating the activity of such genes or treatments that involve alternate pathways, may ameliorate the disease condition.

Generation of Targeting Constructs

The targeting construct of the present invention may be produced using standard methods known in the art. (See, e.g., Sambrook, et al., 1989, Molecular Cloning: A Laboratory Manual, Second Edition, Cold Spring Harbor Laboratory Press, Cold Spring Harbor, N.Y.; E. N. Glover (eds.), 1985, DNA Cloning: A Practical Approach, Volumes I and II; M. J. Gait (ed.), 1984, Oligonucleotide Synthesis; B. D. Hames & S. J. Higgins (eds.), 1985, Nucleic Acid Hybridization; B. D. Hames & S. J. Higgins (eds.), 1984, Transcription and Translation; R. I. Freshney (ed.), 1986, Animal Cell Culture; Immobilized Cells and Enzymes, IRL Press, 1986; B. Perbal, 1984, A Practical Guide To Molecular Cloning; F. M. Ausubel et al., 1994, Current Protocols in Molecular Biology, John Wiley & Sons, Inc.). For example, the targeting construct may be prepared in accordance with conventional ways, where sequences may be synthesized, isolated from natural sources, manipulated, cloned, ligated, subjected to in vitro mutagenesis, primer repair, or the like. At various stages, the joined sequences may be cloned, and analyzed by restriction analysis, sequencing, or the like.

The targeting DNA can be constructed using techniques well known in the art. For example, the targeting DNA may be produced by chemical synthesis of oligonucleotides, nick-translation of a double-stranded DNA template, polymerase chain-reaction amplification of a sequence (or ligase chain reaction amplification), purification of prokaryotic or target cloning vectors harboring a sequence of interest (e.g., a cloned cDNA or genomic DNA, synthetic DNA or from any of the aforementioned combination) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence. Moreover, the length of homology may be selected using known methods in the art. For example, selection may be based on the sequence composition and complexity of the predetermined endogenous target DNA sequence(s).

The targeting construct of the present invention typically comprises a first sequence homologous to a portion or region of the GAMT gene and a second sequence homologous to a second portion or region of the GAMT gene. The targeting construct further comprises a positive selection marker, which is preferably positioned in between the first and the second DNA sequences that are homologous to a portion or region of the target DNA sequence. The positive selection marker may be operatively linked to a promoter and a polyadenylation signal. Other regulatory sequences known in the art may be incorporated into the targeting construct to disrupt or control expression of a particular gene in a specific cell type. In addition, the targeting construct may also include a sequence coding for a screening marker, for example, green fluorescent protein (GFP), or another modified fluorescent protein.

Similarly, a targeting construct of the present invention may also include a sequence homologous to a portion or region of the CRTR gene and a second sequence homologous to a second portion or region of the CRTR gene. The targeting construct may further comprise a positive selection marker, which may be operatively linked to a promoter and a polyadenylation signal, other regulatory sequences and a sequence coding for a screening marker.

Similarly, a targeting construct of the present invention may also include a sequence homologous to a portion or region of the AGAT gene and a second sequence homologous to a second portion or region of the AGAT gene. The targeting construct may further comprise a positive selection marker, which may be operatively linked to a promoter and a polyadenylation signal, other regulatory sequences and a sequence coding for a screening marker.

Although the size of the homologous sequence is not critical and can range from as few as 50 base pairs to as many as 100 kb, preferably each fragment is greater than about 1 kb in length, more preferably between about 1 and about 10 kb, and even more preferably between about 1 and about 5 kb. One of skill in the art will recognize that although larger fragments may increase the number of homologous recombination events in embryonic stem cells, larger fragments will also be more difficult to clone.

Generation of Cells and Confirmation of Homologous Recombination Events

Once an appropriate targeting construct has been prepared, the targeting construct may be introduced into an appropriate host cell using any method known in the art. Various techniques may be employed in the present invention, including, for example, pronuclear microinjection; retrovirus mediated gene transfer into germ lines; gene targeting in embryonic stem cells; electroporation of embryos; sperm-mediated gene transfer; and calcium phosphate/DNA co-precipitates, microinjection of DNA into the nucleus, bacterial protoplast fusion with intact cells, transfection, polycations, e.g., polybrene, polyomithine, etc., or the like (See, e.g., U.S. Pat. No. 4,873,191; Van derPutten, et al., 1985, Proc. Natl. Acad. Sci., USA 82:6148-6152; Thompson, et al., 1989, Cell 56:313-321; Lo, 1983, Mol Cell. Biol. 3:1803-1814; Lavitrano, et al., 1989, Cell, 57:717-723). Various techniques for transforming mammalian cells are known in the art. (See, e.g., Gordon, 1989, Intl. Rev. Cytol., 115:171-229; Keown et al., 1989, Methods in Enzymology; Keown et al., 1990, Methods and Enzymology, Vol. 185, pp. 527-537; Mansour et al., 1988, Nature, 336:348-352).

In a preferred aspect of the present invention, the targeting construct is introduced into host cells by electroporation. In this process, electrical impulses of high field strength reversibly permeabilize biomembranes allowing the introduction of the construct. The pores created during electroporation permit the uptake of macromolecules such as DNA. (See, e.g., Potter, H., et al., 1984, Proc. Nat'l. Acad. Sci. U.S.A. 81:7161-7165).

Any cell type capable of homologous recombination may be used in the practice of the present invention. Examples of such target cells include cells derived from vertebrates including mammals such as humans, bovine species, ovine species, murine species, simian species, and ether eucaryotic organisms such as filamentous fungi, and higher multicellular organisms such as plants. Preferably, the cells of the present invention are derived from murine species.

Preferred cell types include embryonic stem cells, which are typically obtained from pre-implantation embryos cultured in vitro. (See, e.g., Evans, M. J., et al., 1981, Nature 292:154-156; Bradley, M. O., et al., 1984, Nature 309:255-258; Gossler et al., 1986, Proc. Natl. Acad. Sci. USA 83:9065-9069; and Robertson, et al., 1986, Nature 322:445-448). The embryonic stem cells are cultured and prepared for introduction of the targeting construct using methods well known to the skilled artisan. (See, e.g., Robertson, E. J. ed. “Teratocarcinomas and Embryonic Stem Cells, a Practical Approach”, IRL Press, Washington D.C., 1987; Bradley et al., 1986, Current Topics in Devel. Biol. 20:357-371; by Hogan et al., in “Manipulating the Mouse Embryo”: A Laboratory Manual, Cold Spring Harbor Laboratory Press, Cold Spring Harbor N.Y., 1986; Thomas et al., 1987, Cell 51:503; Koller et al., 1991, Proc. Natl. Acad. Sci. USA, 88:10730; Dorin et al., 1992, Transgenic Res. 1:101; and Veis et al., 1993, Cell 75:229). The embryonic stem cells that will be inserted with the targeting construct are derived from an embryo or blastocyst of the same species as the developing embryo into which they are to be introduced. Embryonic stem cells are typically selected for their ability to integrate into the inner cell mass and contribute to the germ line of an individual when introduced into the mammal in an embryo at the blastocyst stage of development. Thus, any embryonic stem cell line having this capability is suitable for use in the practice of the present invention.

The present invention may also be used to knockout genes in cell types, such as stem cells. By way of example, stem cells may be muscle progenitor and precursor cells. These cells, comprising a disruption or knockout of a gene, may be particularly useful in the study of GAMT, CRTR and/or AGAT gene function in individual developmental pathways. Stem cells may be derived from any vertebrate species, such as mouse, rat, dog, cat, pig, rabbit, human, non-human primates and the like. Preferably the stem cells are derived from mouse.

After the targeting construct has been introduced into cells, the cells in which successful gene targeting has occurred are identified. Insertion of the targeting construct into the targeted gene is typically detected by identifying cells for expression of the marker gene. In a preferred embodiment, the cells transformed with the targeting construct of the present invention are subjected to treatment with an appropriate agent that selects against cells not expressing the selectable marker. Only those cells expressing the selectable marker gene survive and/or grow under certain conditions. For example, cells that express the introduced neomycin resistance gene are resistant to the compound G418, while cells that do not express the neo gene marker are killed by G418. If the targeting construct also comprises a screening marker such as GFP, homologous recombination can be identified through screening cell colonies under a fluorescent light. Cells that have undergone homologous recombination will have deleted the GFP gene and will not fluoresce.

If a regulated positive selection method is used in identifying homologous recombination events, the targeting construct is designed so that the expression of the selectable marker gene is regulated in a manner such that expression is inhibited following random integration but is permitted (de-repressed) following homologous recombination. More particularly, the transfected cells are screened for expression of the neo gene, which requires that (1) the cell was successfully electroporated, and (2) lac repressor inhibition of neo transcription was relieved by homologous recombination. This method allows for the identification of transfected cells and homologous recombinants to occur in one step with the addition of a single drug.

Alternatively, a positive-negative selection technique may be used to select homologous recombinants. This technique involves a process in which a first drug is added to the cell population, for example, a neomycin-like drug to select for growth of transfected cells, i.e. positive selection. A second drug, such as FIAU is subsequently added to kill cells that express the negative selection marker, i.e. negative selection. Cells that contain and express the negative selection marker are killed by a selecting agent, whereas cells that do not contain and express the negative selection marker survive. For example, cells with non-homologous insertion of the construct express HSV thymidine kinase and therefore are sensitive to the herpes drugs such as gancyclovir (GANC) or FIAU (1-(2-deoxy 2-fluoro-B-D-arabinofluranosyl)-5-iodouracil). (See, e.g., Mansour et al., Nature 336:348-352: (1988); Capecchi, Science 244:1288-1292, (1989); Capecchi, Trends in Genet. 5:70-76 (1989)).

Successful recombination may be identified by analyzing the DNA of the selected cells to confirm homologous recombination. Various techniques known in the art, such as PCR and/or Southern analysis may be used to confirm homologous recombination events. Homologous recombination may also be used to disrupt genes in stem cells, and other cell types, which are not totipotent embryonic stem cells.

In cells that are not totipotent it may be desirable to knock out both copies of the target using methods that are known in the art. For example, cells comprising homologous recombination at a target locus that have been selected for expression of a positive selection marker (e.g., Neo^{r}) and screened for non-random integration, can be further selected for multiple copies of the selectable marker gene by exposure to elevated levels of the selective agent (e.g., G418). The cells are then analyzed for homozygosity at the target locus. Alternatively, a second construct can be generated with a different positive selection marker inserted between the two homologous sequences. The two constructs can be introduced into the cell either sequentially or simultaneously, followed by appropriate selection for each of the positive marker genes. The final cell is screened for homologous recombination of both alleles of the target.

Production of Transgenic Animals

Selected cells are then injected into a blastocyst (or other stage of development suitable for the purposes of creating a viable animal, such as, for example, a morula) of an animal (e.g., a mouse) to form chimeras (see e.g., Bradley, A. in Teratocarcinomas and Embryonic Stem Cells: A Practical Approach, E. J. Robertson, ed., IRL, Oxford, pp. 113-152 (1987)). Alternatively, selected embryonic stem cells can be allowed to aggregate with dissociated mouse embryo cells to form the aggregation chimera. A chimeric embryo can then be implanted into a suitable pseudopregnant female foster animal and the embryo brought to term. Chimeric progeny harboring the homologously-recombined DNA in their germ cells can be used to breed animals in which all cells of the animal contain the homologously recombined DNA. In one embodiment, chimeric progeny mice are used to generate a mouse with a heterozygous disruption in any one of the GAMT, CRTR and/or AGAT genes. Heterozygous transgenic mice can then be mated. It is well known in the art that typically one-quarter of the offspring of such matings will have a homozygous disruption at least one of these genes.

A recently generated knockout GAMT mouse reportedly has a myopathy and other metabolic problems (Schmidt, A. et al., Human Molecular Genetics, 13(9):905-21 (2004)). Additionally, based on microarray assays, it is known that the GAMT mRNA is down-regulated in human males with DMD. One embodiment of the present invention is a double knockout mouse lacking both dystrophin gene activity and GAMT gene activity that mimics human DMD. This mouse was produced by crossing the GAMT null mouse into the mdx background to produce a double knockout of the GAMT and dystrophin genes which provides a murine model of human DMD. This double-null GAMT:mdx mouse appears clumsy, small, relatively inactive and typically dies by six weeks of age. FIG. 1 shows the histology of leg muscle from a 6 week old double-null GAMT:mdx mouse. These data indicate that the double-null phenotype is more severe than the wild-type or mdx mouse and is similar to the phenotype and histopathology of human DMD. Thus, this double knockout mouse is presently the best mouse model of human DMD, both clinically and histopathologically. This mouse model truly reflects the transcriptional profile shown in males with DMD which is an absent dystrophin protein and downregulated GAMT expression. This double null mouse provides a novel experimental reagent to test treatments and cures for human DMD including main-stream dystrophin gene repair/transfer. These double knockout animals may also be used to test and prove interventions in this mouse before doing human clinical trials.

Heterozygous and homozygous transgenic mice can be compared to normal, wild type mice to determine whether disruption of any of the GAMT, CRTR and/or AGAT gene causes phenotypic changes resembling DMD in human males, especially pathological changes. For example, heterozygous and homozygous mice may be evaluated for phenotypic changes by physical examination, necropsy, histology, clinical chemistry, complete blood count, body weight, organ weights, and cytological evaluation of bone marrow.

Conditional Transgenic Animals

The present invention further contemplates conditional transgenic or knockout animals, such as those produced using recombination methods. Bacteriophage P1 Cre recombinase and flp recombinase from yeast plasmids are two non-limiting examples of site-specific DNA recombinase enzymes that cleave DNA at specific target sites (lox P sites for cre recombinase and frt sites for flp recombinase) and catalyze a ligation of this DNA to a second cleaved site. A large number of suitable alternative site-specific recombinases have been described, and their genes can be used in accordance with the method of the present invention. Such recombinases include the Int recombinase of bacteriophage λ (with or without X is) (Weisberg, R. et al., in Lambda II, (Hendrix, R., et al., Eds.), Cold Spring Harbor Press, Cold Spring Harbor, N.Y., pp. 211-50 (1983), herein incorporated by reference); TpnI and the P-lactamase transposons (Mercier, et al., J. Bacteriol., 172:3745-57 (1990)); the Tn3 resolvase (Flanagan & Fennewald J. Molec. Biol., 206:295-304 (1989); Stark, et al., Cell, 58:779-90 (1989)); the yeast recombinases (Matsuzaki, et al., J. Bacteriol., 172:610-18 (1990)); the B. subtilis SpoIVC recombinase (Sato, et al., J. Bacteriol. 172:1092-98 (1990)); the Flp recombinase (Schwartz & Sadowski, J. Molec. Biol., 205:647-658 (1989); Parsons, et al., J. Biol. Chem., 265:4527-33 (1990); Golic & Lindquist, Cell, 59:499-509 (1989); Amin, et al., J. Molec. Biol., 214:55-72 (1990)); the Hin recombinase (Glasgow, et al., J. Biol. Chem., 264:10072-82 (1989)); immunoglobulin recombinases (Malynn, et al., Cell, 54:453-460 (1988)); and the Cin recombinase (Haffter & Bickle, EMBO J., 7:3991-3996 (1988); Hubner, et al., J. Molec. Biol., 205:493-500 (1989)), all herein incorporated by reference. Such systems are discussed by Echols (J. Biol. Chem. 265:14697-14700 (1990)); de Villartay (Nature, 335:170-74 (1988)); Craig, (Ann. Rev. Genet., 22:77-105 (1988)); Poyart-Salmeron, et al., (EMBO J. 8:2425-33 (1989)); Hunger-Bertling, et al., (Mol Cell. Biochem., 92:107-16 (1990)); and Cregg & Madden (Mol. Gen. Genet., 219:320-23 (1989)), all herein incorporated by reference.

Cre has been purified to homogeneity, and its reaction with the loxP site has been extensively characterized (Abremski & Hess J. Mol. Biol. 259:1509-14 (1984), herein incorporated by reference). Cre protein has a molecular weight of 35,000 and can be obtained commercially from New England Nuclear/Du Pont. The cre gene (which encodes the Cre protein) has been cloned and expressed (Abremski, et al., Cell 32:1301-11 (1983), herein incorporated by reference). The Cre protein mediates recombination between two loxP sequences (Sternberg, et al., Cold Spring Harbor Symp. Quant. Biol. 45:297-309 (1981)), which may be present on the same or different DNA molecule. Because the internal spacer sequence of the loxP site is asymmetrical, two loxP sites can exhibit directionality relative to one another (Hoess & Abremski Proc. Natl. Acad. Sci. U.S.A. 81:1026-29 (1984)). Thus, when two sites on the same DNA molecule are in a directly repeated orientation, Cre will excise the DNA between the sites (Abremski, et al., Cell 32:1301-11 (1983)). However, if the sites are inverted with respect to each other, the DNA between them is not excised after recombination but is simply inverted. Thus, a circular DNA molecule having two loxP sites in direct orientation will recombine to produce two smaller circles, whereas circular molecules having two loxP sites in an inverted orientation simply invert the DNA sequences flanked by the loxP sites. In addition, recombinase action can result in reciprocal exchange of regions distal to the target site when targets are present on separate DNA molecules.

Recombinases have important application for characterizing gene function in knockout models. For example, when the constructs described herein are used to disrupt GAMT genes, a fusion transcript can be produced when insertion of the positive selection marker occurs downstream (3′) of the translation initiation site of the GAMT gene. The fusion transcript could result in some level of protein expression with unknown consequence. It has been suggested that insertion of a positive selection marker gene can affect the expression of nearby genes. These effects may make it difficult to determine gene function after a knockout event since one could not discern whether a given phenotype is associated with the inactivation of a gene, or the transcription of nearby genes. Both potential problems are solved by exploiting recombinase activity. When the positive selection marker is flanked by recombinase sites in the same orientation, the addition of the corresponding recombinase will result in the removal of the positive selection marker. In this way, effects caused by the positive selection marker or expression of fusion transcripts are avoided.

In one embodiment, purified recombinase enzyme is provided to the cell by direct microinjection. In another embodiment, recombinase is expressed from a co-transfected construct or vector in which the recombinase gene is operably linked to a functional promoter. An additional aspect of this embodiment is the use of tissue-specific or inducible recombinase constructs that allow the choice of when and where recombination occurs. One method for practicing the inducible forms of recombinase-mediated recombination involves the use of vectors that use inducible or tissue-specific promoters or other gene regulatory elements to express the desired recombinase activity. The inducible expression elements are preferably operatively positioned to allow the inducible control or activation of expression of the desired recombinase activity. Examples of such inducible promoters or other gene regulatory elements include, but are not limited to, tetracycline, metallothionine, ecdysone, and other steroid-responsive promoters, rapamycin responsive promoters, and the like (No, et al., Proc. Natl. Acad. Sci. USA, 93:3346-51 (1996); Furth, et al., Proc. Natl. Acad. Sci. USA, 91:9302-6 (1994)). Additional control elements that can be used include promoters requiring specific transcription factors such as viral, promoters. Vectors incorporating such promoters only express recombinase activity in cells that express the necessary transcription factors. Using these constructs, it is possible to study and exploit the differences in the human and mouse regulatory elements of the GAMT gene in administering treatments of the muscular dystrophies in non-murine patients.

The cell- and animal-based systems described herein can be utilized as models for muscular dystrophy diseases. Animals of any species, including, but not limited to, mice, rats, rabbits, guinea pigs, pigs, micro-pigs, goats, and non-human primates, e.g., baboons, monkeys, and chimpanzees may be used to generate disease animal models. In addition, cells from humans may be used. These systems may be used in a variety of applications. Such assays may be utilized as part of screening strategies designed to identify agents, such as compounds that are capable of ameliorating symptoms of muscular dystrophies. Thus, the animal- and cell-based models may be used to identify drugs, pharmaceuticals, therapies and interventions that may be effective in treating DMD.

Cell-based systems may be used to identify compounds that may act to ameliorate DMD symptoms. For example, such cell systems may be exposed to a compound suspected of exhibiting an ability to ameliorate symptoms of DMD, at a sufficient concentration and for a time sufficient to elicit such an amelioration of DMD symptoms in the exposed cells. After exposure, the cells are examined to determine whether one or more of the cellular phenotypes of DMD has been altered to resemble a more normal or more wild type, non-disease phenotype.

In addition, animal-based DMD model systems, such as those described herein, may be used to identify compounds capable of ameliorating DMD symptoms. Such animal models may be used as test substrates for the identification of drugs, pharmaceuticals, therapies, and interventions that may be effective in treating DMD or other phenotypic characteristics of the animal. For example, animal models may be exposed to a compound or agent suspected of exhibiting an ability to ameliorate disease symptoms, at a sufficient concentration and for a time sufficient to elicit such an amelioration of disease symptoms in the exposed animals. The response of the animals to the exposure may be monitored by assessing the reversal of disorders associated with DMD. Exposure may involve treating mother animals during gestation of the model animals described herein, thereby exposing embryos or fetuses to the compound or agent that may prevent or ameliorate the DMD or symptoms thereof. Neonatal, juvenile, and adult animals can also be exposed.

More particularly, using the animal models of the invention, specifically, transgenic mice or cells derived from these transgenic animals, methods of identifying agents on the basis of their ability to affect at least one phenotype associated with a disruption in the dystrophin, GAMT, CRTR and/or AGAT genes. In one embodiment, the present invention provides a method of identifying agents having an effect on GAMT expression or function. In another embodiment, the present invention provides a method of identifying agents having an effect on CRTR expression or function. In yet another embodiment, the present invention provides a method of identifying agents having an effect on AGAT expression or function. The agents identified may modulate the expression of one or more of these genes by increasing its expression, decreasing its expression, increasing or decreasing the activity of the respective gene product or (especially with respect to the creatine transporter protein) by altering the localization of the gene product within the cell or on the cell membrane. The method includes measuring a physiological response of the animal or a biological or metabolic response of the cell, to the agent, and comparing the response to a control animal or cell, wherein the response of the animal or cell having an alteration in one of the test genes as compared to the control animal indicates the specificity of the agent. A “physiological response” is any biological or physical parameter of an animal that can be measured. Molecular assays (e.g., gene transcription, protein production and degradation rates), physical parameters (e.g., exercise physiology tests, measurement of various parameters of respiration, measurement of heart rate or blood pressure, measurement of bleeding time) and cellular assays (e.g., immunohistochemical assays of cell surface markers, or the ability of cells to aggregate or proliferate) can be used to assess a physiological response. The transgenic animals and cells of the present invention may be utilized as models for diseases, disorders, or conditions associated with phenotypes relating to a disruption in the dystrophin, GAMT, CRTR and/or AGAT genes.

The present invention provides unique animal and cell models for testing and developing new treatments relating to the behavioral phenotypes of DMD. Analysis of the behavioral phenotype allows for the development of an animal model useful for testing the efficacy of proposed genetic and pharmacological therapies for genetic diseases such as muscular dystrophies and, in particular, DMD.

GAMT/CRTR/AGAT Gene Products

The present invention further contemplates use of GAMT, CRTR and/or AGAT gene sequences to produce GAMT, CRTR and/or AGAT gene products. Such gene products may include proteins that represent functionally equivalent gene products. Such an equivalent gene product may contain deletions, additions or substitutions of amino acid residues within the amino acid sequence encoded by the gene sequences described herein, but which result in a silent change, thereby producing a functionally equivalent GAMT, CRTR and/or AGAT gene product. Amino acid substitutions may be made on the basis of similarity in polarity, charge, solubility, hydrophobicity, hydrophilicity, and/or the amphipathic nature of the residues involved.

For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Functionally equivalent”, as utilized herein, refers to a protein capable of exhibiting a substantially similar in vivo activity as the endogenous gene products encoded by the GAMT, CRTR or AGAT sequences. Alternatively, when utilized as part of an assay, “functionally equivalent” may refer to peptides capable of interacting with other cellular or extracellular molecules in a manner substantially similar to the way in which the corresponding portion of the endogenous gene product would.

Other protein products useful according to the methods of the invention are peptides derived from, or based on, the GAMT, CRCT and/or AGAT genes produced by recombinant or synthetic means (derived peptides).

These gene products may be produced by recombinant DNA technology using techniques well known in the art. Thus, methods for preparing the gene polypeptides and peptides of the invention by expressing nucleic acid encoding gene sequences are described herein. Methods that are well known to those skilled in the art can be used to construct expression vectors containing gene protein coding sequences and appropriate transcriptional/translational control signals. These methods include, for example, in vitro recombinant DNA techniques, synthetic techniques and in vivo recombination/genetic recombination (See, e.g., Sambrook, et al., 1989, supra, and Ausubel, et al., 1989, supra). Alternatively, RNA capable of encoding gene protein sequences may be chemically synthesized using, for example, automated synthesizers (See, e.g. Oligonucleotide Synthesis: A Practical Approach, Gait, M. J. ed., IRL Press, Oxford (1984)).

A variety of host-expression vector systems may be utilized to express the gene coding sequences of the invention. Such host-expression systems represent vehicles by which the coding sequences of interest may be produced and subsequently purified, but also represent cells that may, when transformed or transfected with the appropriate nucleotide coding sequences, exhibit the gene protein of the invention in situ. These include, but are not limited to, microorganisms such as bacteria (e.g., E. coli, B. subtilis) transformed with recombinant bacteriophage DNA, plasmid DNA or cosmid DNA expression vectors containing gene protein coding sequences; yeast (e.g. Saccharomyces, Pichia) transformed with recombinant yeast expression vectors containing the gene protein coding sequences; insect cell systems infected with recombinant virus expression vectors (e.g., baculovirus) containing the gene protein coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing gene protein coding sequences; or mammalian cell systems (e.g. COS, CHO, BHK, 293, 3T3) harboring recombinant expression constructs containing promoters derived from the genome of mammalian cells (e.g., metallothionine promoter) or from mammalian viruses (e.g., the adenovirus late promoter; the vaccinia virus 7.5 K promoter).

In bacterial systems, a number of expression vectors may be advantageously selected depending upon the use intended for the gene protein being expressed. For example, when a large quantity of such a protein is to be produced, for the generation of antibodies or to screen peptide libraries, for example, vectors that direct the expression of high levels of fusion protein products that are readily purified may be desirable. Such vectors include, but are not limited to, the E. coli expression vector pUR278 (Ruther et al., EMBO J., 2:1791-94 (1983)), in which the gene protein coding sequence may be ligated individually into the vector in frame with the lac Z coding region so that a fusion protein is produced; pIN vectors (Inouye & Inouye, Nucleic Acids Res., 13:3101-09 (1985); Van Heeke et al., J. Biol. Chem., 264:5503-9 (1989)); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned GAMT gene protein can be released from the GST moiety.

In an insect system, Autographa californica nuclear polyhedrosis virus (AcNPV) maybe used as a vector to express foreign genes. The virus grows in Spodoptera frugiperda cells. The gene coding sequence may be cloned individually into non-essential regions (for example the polyhedrin gene) of the virus and placed under control of an AcNPV promoter (for example the polyhedrin promoter). Successful insertion of gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (See, e.g., Smith, et al., J. Virol. 46: 584-93 (1983); U.S. Pat. No. 4,745,051).

In mammalian host cells, a number of viral-based expression systems may be utilized. In cases where an adenovirus is used as an expression vector, the gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing gene protein in infected hosts. (e.g., see Logan et al., Proc. Natl. Acad. Sci. USA, 81:3655-59 (1984)). Specific initiation signals may also be required for efficient translation of inserted gene coding sequences. These signals include the ATG initiation codon and adjacent sequences. In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter, et al., Methods in Enzymol., 153:516-44 (1987)).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include, but are not limited to, CHO, VERO, BHK, HeLa, COS, MDCK, 293, 3T3, and W138.

For long-term, high-yield production of recombinant proteins, stable expression is preferred. For example, cell lines that stably express the gene protein may be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells that stably integrate the plasmid into their chromosomes and grow, to form foci, which in turn can be cloned and expanded into cell lines. This method may advantageously be used to engineer cell lines that express the gene protein. Such engineered cell lines may be particularly useful in screening and evaluation of compounds that affect the endogenous activity of the gene protein.

In a preferred embodiment, timing and/or quantity of expression of the recombinant protein can be controlled using an inducible expression construct. Inducible constructs and systems for inducible expression of recombinant proteins will be well known to those skilled in the art. Examples of such inducible promoters or other gene regulatory elements include, but are not limited to, tetracycline, metallothionine, ecdysone, and other steroid-responsive promoters, rapamycin responsive promoters, and the like (No, et al., Proc. Natl. Acad. Sci. USA, 93:3346-51 (1996); Furth, et al., Proc. Natl. Acad. Sci. USA, 91:9302-6 (1994)). Additional control elements that can be used include promoters requiring specific transcription factors such as viral, particularly HIV, promoters. In one in embodiment, a Tet inducible gene expression system is utilized. (Gossen et al., Proc. Natl. Acad. Sci. USA, 89:5547-51 (1992); Gossen, et al., Science, 268:1766-69 (1995)). Tet Expression Systems are based on two regulatory elements derived from the tetracycline-resistance operon of the E. coli Tn10 transposon—the tetracycline repressor protein (TetR) and the tetracycline operator sequence (tetO) to which TetR binds. Using such a system, expression of the recombinant protein is placed under the control of the tetO operator sequence and transfected or transformed into a host cell. In the presence of TetR, which is co-transfected into the host cell, expression of the recombinant protein is repressed due to binding of the TetR protein to the tetO regulatory element. High-level, regulated gene expression can then be induced in response to varying concentrations of tetracycline (Tc) or Tc derivatives such as doxycycline (Dox), which compete with tetO elements for binding to TetR. Constructs and materials for tet inducible gene expression are available commercially from CLONTECH Laboratories, Inc., Palo Alto, Calif.

When used as a component in an assay system, the gene protein may be labeled, either directly or indirectly, to facilitate detection of a complex formed between the gene protein and a test substance. Any of a variety of suitable labeling systems may be used including but not limited to radioisotopes such as ¹²⁵I; enzyme labeling systems that generate a detectable calorimetric signal or light when exposed to substrate; and fluorescent labels. Where recombinant DNA technology is used to produce the gene protein for such assay systems, it may be advantageous to engineer fusion proteins that can facilitate labeling, immobilization and/or detection.

Indirect labeling involves the use of a protein, such as a labeled antibody, which specifically binds to the gene product. Such antibodies include, but are not limited to, polyclonal, monoclonal, chimeric, single chain, Fab fragments and fragments produced by a Fab expression library.

Described herein are methods for the production of antibodies capable of specifically recognizing one or more epitopes. Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of the dystrophin, GAMT, CRTR and/or AGAT genes in a biological sample, or, alternatively, as a method for the inhibition of abnormal activity of one or more of these genes or the proteins encoded by these genes. Additionally, the homology of the mouse and human GAMT is at least 86% identical and therefore, antibody probes will cross-react across species of mouse, human and likely canine. Thus, such antibodies may be utilized as part of disease treatment methods, and/or may be used as part of diagnostic techniques whereby patients may be tested for abnormal levels of dystrophin, GAMT, CRTR and/or AGAT proteins, or for the presence of abnormal forms of such proteins.

For the production of antibodies, various host animals may be immunized by injection with a gene, its expression product or a portion thereof. Such host animals may include, but are not limited to, rabbits, mice, rats, goats and chickens. Various adjuvants may be used to increase the immunological response, depending on the host species, including. but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as the GAMT gene product, or an antigenic functional derivative thereof. For the production of polyclonal antibodies, host animals such as those described above, may be immunized by injection with a gene product supplemented with adjuvants as described above.

Monoclonal antibodies, which are homogeneous populations of antibodies to a particular antigen, may be obtained by any technique that provides for the production of antibody molecules by continuous cell lines in culture. These include, but are not limited to the hybridoma technique of Kohler and Milstein, Nature, 256:495-7 (1975); and U.S. Pat. No. 4,376,110), the human B-cell hybridoma technique (Kosbor, et al., Immunology Today, 4:72 (1983); Cote, et al., Proc. Natl. Acad. Sci. USA, 80:2026-30 (1983)), and the EBV-hybridoma technique (Cole, et al., in Monoclonal Antibodies And Cancer Therapy, Alan R. Liss, Inc., New York, pp. 77-96 (1985)). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison, et al., Proc. Natl. Acad. Sci., 81:6851-6855 (1984); Takeda, et al., Nature, 314:452-54 (1985)) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, Science 242:423-26 (1988); Huston, et al., Proc. Natl. Acad. Sci. USA, 85:5879-83 (1988); and Ward, et al., Nature, 334:544-46 (1989)) can be adapted to produce gene-single chain antibodies. Single chain antibodies are typically formed by linking the heavy and light chain fragments of the Fv region via an amino acid bridge, resulting in a single chain polypeptide.

Antibody fragments that recognize specific epitopes may be generated by known techniques. For example, such fragments include, but are not limited to, the F(ab′)₂ fragments that can be produced by pepsin digestion of the antibody molecule and the Fab fragments that can be generated by reducing the disulfide bridges of the F(ab′)₂ fragments. Alternatively, Fab expression libraries may be constructed (Huse, et al., Science, 246:1275-81 (1989)) to allow rapid and easy identification of monoclonal Fab fragments with the desired specificity.

Screening Methods

The present invention may be employed in a process for screening for agents such as agonists, i.e. agents that bind to and activate GAMT, CRTR and/or AGAT polypeptides, or antagonists, i.e. agents that inhibit the activity or interaction of GAMT, CRTR and/or AGAT polypeptides with their respective ligands. Thus, polypeptides of the invention may also be used to assess the binding of small molecule substrates and ligands in, for example, cells, cell-free preparations, chemical libraries, and natural product mixtures as known in the art. Any methods routinely used to identify and screen for agents that can modulate receptors or regulatory DNA binding elements may be used in accordance with the present invention.

Using these assays, the present invention provides methods for identifying and screening for agents that modulate expression or function of GAMT, CRTR or AGAT. More particularly, cells that contain and express GAMT, CRTR and/or AGAT gene sequences may be used to screen for therapeutic agents. The therapeutic agents may include agents that modulate expression of the GAMT, CRTR or AGAT genes by increasing their expression, decreasing their expression, inhibiting or enhancing their repression, interacting with regulatory elements that effect one or more of these genes, increasing or decreasing the stability of their transcribed RNA messages, binding to one of their transcribed RNA messages, decreasing or increasing the translation of one of their RNA messages, or stabilizing or destabilizing their respective gene products.

Cells that contain and express GAMT, CRTR and/or AGAT gene sequences may include non-recombinant monocyte cell lines, such as U937 (ATCC# CRL-1593), THP-1 (ATCC# TIB-202), and P388D1 (ATCC# TIB-63); endothelial cells such as HUVEC's and bovine aortic endothelial cells (BAEC's); as well as generic mammalian cell lines such as HeLa cells and COS cells, e.g., COS-7 (ATCC# CRL-1651). Further, such cells may include recombinant, transgenic cell lines. For example, the transgenic mice of the invention may be used to generate cell lines, containing one or more cell types involved in DMD, that can be used as cell culture models for that muscle disorder. While cells, tissues, and primary cultures derived from the disease transgenic animals of the invention may be utilized, the generation of continuous cell lines is preferred. For examples of techniques that may be used to derive a continuous cell line from the transgenic animals, see Small, et al., Mol. Cell Biol., 5:642-48 (1985).

GAMT, CRTR and/or AGAT gene sequences may be introduced into, and overexpressed in, the genome of the cell of interest. In order to overexpress the gene sequence of interest, the coding portion of the gene sequence may be ligated to a regulatory sequence that is capable of driving gene expression in the cell type of interest. Such regulatory regions will be well known to those of skill in the art, and may be utilized in the absence of undue experimentation. GAMT, CRTR and/or AGAT gene sequences may also be disrupted or underexpressed. Cells having disruptions in, or underexpressed, GAMT, CRTR and/or AGAT gene sequences may be used, for example, to screen for agents capable of affecting alternative pathways that compensate for any loss of function attributable to the disruption or underexpression.

Patients displaying or at risk for developing a muscular dystrophy may be treated by gene therapy. One or more copies of normal GAMT, CRTR and/or AGAT genes, or a portion of these genes that directs the production of a normal protein with wild-type gene function, may be upregulated or inserted into the patient's cells using vectors that include, but are not limited to adenovirus, adeno-associated virus, and retrovirus vectors, in addition to other particles that introduce DNA into cells, such as liposomes. Additionally, techniques such as those described above may be utilized for the introduction of normal GAMT, CRTR and/or AGAT gene sequences into human cells.

In vitro systems may be designed to identify compounds capable of binding the GAMT, CRTR and/or AGAT gene products. Such compounds may include, but are not limited to, peptides made of D-and/or L-configuration amino acids (in, for example, the form of random peptide libraries; (see e.g., Lam, et al., Nature, 354:82-4 (1991)), phosphopeptides (in, for example, the form of random or partially degenerate, directed phosphopeptide libraries; See, e.g., Songyang, et al., Cell, 72:767-78 (1993)), antibodies, and small organic or inorganic molecules. Compounds identified may be useful, for example, in modulating the activity of GAMT gene proteins, preferably mutant GAMT gene proteins; elaborating the biological function of the GAMT gene protein; or screening for compounds that disrupt normal GAMT gene interactions or themselves disrupt such interactions.

The principle of the assays used to identify compounds that bind to the GAMT, CRTR and/or AGAT proteins involves preparing a reaction mixture of the respective protein and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring either or both proteins or the test substance onto a solid phase and detecting target protein/test substance complexes anchored on the solid phase at the end of the reaction. In one embodiment of such a method, the GAMT, CRTR and/or AGAT proteins may be anchored onto a solid surface, and the test compound, which is not anchored, may be labeled, either directly or indirectly.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for GAMT, CRTR and/or AGAT gene products or the test compound to anchor any complexes formed in solution, and a labeled antibody specific for the other component of the possible complex to detect anchored complexes.

Compounds that are shown to bind to a particular gene product through one of the methods described above can be further tested for their ability to elicit a biochemical response from interaction with the respective GAMT, CRTR and/or AGAT proteins. Agonists, antagonists and/or inhibitors of the expression product can be identified utilizing assays well known in the art. Additionally, analogs of the expression products that act to mimic the effect of the wild type gene products may be identified.

Antisense, Ribozymes, and Antibodies

Other agents that may be used as therapeutics include the GAMT, CRTR and/or AGAT genes, their expression products and functional fragments thereof. Additionally, agents that reduce or inhibit mutant GAMT gene activity may be used to ameliorate DMD symptoms. Such agents include antisense, ribozyme, and triple helix molecules. Techniques for the production and use of such molecules are well known to those of skill in the art.

Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the GAMT gene nucleotide sequence of interest, are preferred.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage. The composition of ribozyme molecules must include one or more sequences complementary to at lest one of dystrophin, GMAT, CRTR or AGAT mRNA, and must include the well known catalytic sequence responsible for mRNA cleavage. For this sequence, see U.S. Pat. No. 5,093,246, which is incorporated by reference herein in its entirety. As such, within the scope of the invention are engineered hammerhead motif ribozyme molecules that specifically and efficiently catalyze endonucleolytic cleavage of RNA sequences encoding dystrophin, GAMT, CRTR and/or AGAT proteins.

Specific ribozyme cleavage sites within any potential RNA target are initially identified by scanning the molecule of interest for ribozyme cleavage sites that include the following sequences, GUA, GWU and GUC. Once identified, short RNA sequences of between 15 and 20 ribonucleotides corresponding to the region of the gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

It is possible that the antisense, ribozyme, and/or triple helix molecules described herein may reduce or inhibit the transcription (triple helix) and/or translation (antisense, ribozyme) of mRNA produced by both normal and mutant GAMT, CRTR and/or AGAT gene alleles. In order to ensure that substantially normal levels of gene activity are maintained, nucleic acid molecules that encode and express gene polypeptides exhibiting normal activity may be introduced into cells that do not contain sequences susceptible to whatever antisense, ribozyme, or triple helix treatments are being utilized. Alternatively, it may be preferable to coadminister normal gene proteins into the cell or tissue in order to maintain the requisite level of cellular or tissue gene activity.

Anti-sense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as, for example, solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Antibodies that are both specific for the GAMT protein, and in particular, mutant gene protein, and interfere with its activity may be used to inhibit GMAT or mutant GAMT gene function. Such antibodies may be generated against the proteins themselves or against peptides corresponding to portions of the proteins using standard techniques known in the art and as also described herein. Such antibodies include, but are not limited to, polyclonal, monoclonal, Fab fragments, single chain antibodies and chimeric antibodies. For example, antibodies to GAMT have been generated and are described in Lee, H., et al., Biol. Reprod. 50(1):152-62 (1994), and Schmidt, A., et al., Hum. Mol. Genet., 13(9):905-21 (2004).

In instances where the GAMT gene protein is intracellular and whole antibodies are used, internalizing antibodies may be preferred. However, lipofectin liposomes may be used to deliver the antibody or a fragment of the Fab region that binds to the GAMT gene epitope into cells. Where fragments of the antibody are used, the smallest inhibitory fragment that binds to the target or expanded target protein's binding domain is preferred. For example, peptides having an amino acid sequence corresponding to the domain of the variable region of the antibody that binds to the GAMT gene protein may be used. Such peptides may be synthesized chemically or produced via recombinant DNA technology using methods well known in the art (See, e.g., Creighton, Proteins: Structures and Molecular Principles (1984) W. H. Freeman, New York 1983, supra; and Sambrook, et al., 1989, supra). Alternatively, single chain neutralizing antibodies that bind to intracellular GAMT gene epitopes may also be administered. Such single chain antibodies may be administered, for example, by expressing nucleotide sequences encoding single-chain antibodies within the target cell population by utilizing, for example, techniques such as those described in Marasco, et al., Proc. Natl. Acad. Sci. USA, 90:7889-93 (1993).

RNA sequences encoding GAMT, CRTR and/or AGAT proteins may be directly administered to a patient exhibiting DMD symptoms, at a concentration sufficient to produce a level of GAMT proteins such that DMD symptoms are ameliorated. Patients may be treated by gene therapy. One or more copies of a normal GAMT gene, or a portion of the gene that directs the production of a normal GAMT protein with GAMT gene function, may be inserted into cells using vectors that include, but are not limited to adenovirus, adeno-associated virus, and retrovirus vectors, in addition to other particles that introduce DNA into cells, such as liposomes. Additionally, techniques such as those described above may be utilized for the introduction of normal GAMT gene sequences into human cells.

Cells, preferably autologous cells, containing normal gene-expressing gene sequences may then be introduced or reintroduced into the patient at positions that allow for the amelioration of DMD or DMD symptoms.

Pharmaceutical Compositions, Effective Dosages, and Routes of Administration

The identified compounds that modulate gene expression of dystrophin, GAMT/AGAT/CRTR, or the synthesis and/or activity of the corresponding enzymes may be administered to a patient at therapeutically effective doses to treat or ameliorate DMD. A therapeutically effective dose refers to that amount of the compound sufficient to result in amelioration of muscle weakness symptoms in male mammals with DMD.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present invention may be formulated in conventional manner using one or more physiologically-acceptable carriers or excipients. Thus, the compounds and their physiologically-acceptable salts and solvates may be formulated for administration by inhalation or insufflation (either through the mouth or the nose) or oral, buccal, parenteral, topical, subcutaneous, intraperitoneal, intraveneous, intrapleural, intraoccular, intraarterial, or rectal administration. It is also contemplated that pharmaceutical compositions may be administered with other products that potentiate the activity of the compound and optionally, may include other therapeutic ingredients.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the treatments may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulation agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

Oral ingestion is possibly the easiest method of taking any medication, especially for children and babies. Such a route of administration, is generally simple and straightforward and is frequently the least inconvenient or unpleasant route of administration from the patient's point of view. However, this involves passing the material through the stomach, which is a hostile environment for many materials, including proteins and other biologically active compositions. As the acidic, hydrolytic and proteolytic environment of the stomach has evolved efficiently to digest proteinaceous materials into amino acids and oligopeptides for subsequent anabolism, it is hardly surprising that very little or any of a wide variety of biologically active proteinaceous material, if simply taken orally, would survive its passage through the stomach to be taken up by the body in the small intestine. The result, is that many proteinaceous medicaments must be taken in through another method, such as parenterally, often by subcutaneous, intramuscular or intravenous injection.

Pharmaceutical compositions may also include various buffers (e.g., Tris, acetate, phosphate), solubilizers (e.g., Tween, Polysorbate), carriers such as human serum albumin, preservatives (thimerosol, benzyl alcohol) and anti-oxidants such as ascorbic acid in order to stabilize pharmaceutical activity. The stabilizing agent may be a detergent, such as tween-20, tween-80, NP-40 or Triton X-100. EBP may also be incorporated into particulate preparations of polymeric compounds for controlled delivery to a patient over an extended period of time. A more extensive survey of components in pharmaceutical compositions is found in Remington's Pharmaceutical Sciences, 18th ed., A. R. Gennaro, ed., Mack Publishing, Easton, Pa. (1990).

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may, for example, comprise metal or plastic foil, such as a blister pack. The pack or dispenser device may be accompanied by instructions for administration.

In one embodiment, the pharmaceutical compositions of the present invention are formulated to deliver creatine to a patient suffering a muscular dystrophy to achieve creatine uptake in muscle cells with an impaired or absent CRTR. As used herein, “creatine” refers to creatine, physiologically-acceptable salts, derivative and/or analogs thereof. The creatine may be administered in a dosage range of between about 0.1 mg/kg to about 200 mg/kg per patient, per day in single or divided doses. Preferably, the creatine is administered orally in one or more solid or liquid oral formulations. These formulations may include, or may be administered simultaneously with, agents that increase the absorption of creatine. In a related embodiment, analogs of creatine are delivered to a to a patient suffering a muscular dystrophy to achieve creatine-like effects in the muscle cells of the patient. These analogs preferably by-pass the need for an effective or local creatine transporter protein for uptake into muscle cells.

In another preferred embodiment, the pharmaceutical compositions of the present invention are formulated to deliver a target enzyme involved in creatine production such as L-arginine:glycine amidinotransferase, guanidinoacetate methyltransferase and/or the creatine transporter protein, to a patient suffering a muscular dystrophy. The proteins or compounds may be administered by any acceptable route of administration. Preferably, the agent(s) are administered intravenously or intramuscularly in a therapeutically-effective dosage in single or divided doses. The pharmaceutical formulations used to administer the proteins or compounds to the mammal in need of such treatment may include agents that increase the absorption of proteins or inhibit the digestion or hydrolysis of these proteins resulting in elevated creatine levels in the intracellular compartment of muscle fibers and cells, and not just in the blood serum.

Diagnostics

A variety of methods may be employed to diagnose disease conditions associated with GAMT, CRTR and/or AGAT genes. Specifically, reagents may be used, for example, for the detection of the presence of GAMT gene mutations, or the detection of either over- or under-expression of GAMT, CRTR and/or AGAT mRNA, or GAMT/AGAT protein in serum.

According to the diagnostic and prognostic methods of the present invention, alteration of the wild-type GAMT, CRTR and/or AGAT gene locus is detected. In addition, the method can be performed by detecting the wild-type GAMT gene locus and confirming the lack of a predisposition for severe DMD phenotype. “Alteration of a wild-type gene” encompasses all forms of mutations including deletions, insertions and point mutations in the coding and noncoding regions. Deletions may be of the entire gene or only a portion of the gene. Point mutations may result in stop codons, frameshift mutations or amino acid substitutions. Somatic mutations are those that occur only in certain tissues, but are not inherited in the germline. Germline mutations can be found in any bodily tissues and are inherited. If only a single allele is mutated, a heterozygous state is indicated. However, if both alleles are mutated, then an expectation of full DMD is indicated. The finding of gene mutations or altered intramuscular creatine levels by nuclear magnetic resonance (NMR) spectroscopy thus provide both diagnostic and prognostic information. A gene allele that is not deleted (e.g., that found on the sister chromosome to a chromosome carrying the GAMT, CRTR and/or AGAT gene deletion) can be screened for other mutations, such as insertions, small deletions, and point mutations. Mutations leading to non-functional gene products may also be linked to a muscular dystrophy. Point mutational events may occur in regulatory regions, such as in the promoter of the gene, leading to loss or diminution of expression of the mRNA. Point mutations may also abolish proper RNA processing, leading to loss of expression of the GAMT, CRTR and/or AGAT gene products, or a decrease in mRNA stability or translation efficiency.

Microarray analysis of muscle biopsies of boys with DMD shows an expression profile of downregulated GAMT in the absence of dystrophin protein. This represents an in vivo means to test the role of GAMT protein in compensating for cellular challenges produced by a dystrophin-deficiency. Thus, one embodiment of the present invention is a method of assessing the GAMT expression in a mammal by microarray analysis of muscle biopsies taken from the mammal. These tests may be conducted on a mammal suspected of having a muscular dystrophy or a mammal known to have a muscular dystrophy in order to ascertain the role of GAMT and/or creatine in the physiology of the muscular dystrophy in the mammal.

The methods described herein may be performed, for example, by utilizing pre-packaged diagnostic kits comprising at least one specific gene nucleic acid or anti-gene antibody reagent described herein, which may be conveniently used, e.g., in clinical settings, to diagnose patients exhibiting early DMD symptoms or at risk for developing DMD. Any cell type or tissue, preferably platelets, neutrophils or lymphocytes, in which the gene is expressed may be utilized in the diagnostic testing. The GAMT/AGAT/CRTR genes may not be expressed in males with severe DMD and may be partially upregulated in males with mild DMD (Beckers) muscular dystrophy, similar to the mdx mouse.

DNA or RNA from the cell type or tissue to be analyzed may easily be isolated using procedures that are well known to those in the art. Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, PCR In Situ Hybridization: Protocols and Applications, Raven Press, N.Y. (1992).

Gene nucleotide sequences, either RNA or DNA, may, for example, be used in hybridization or amplification assays of biological samples to detect DMD-related gene structures and expression. Such assays may include, but are not limited to, Southern or Northern analyses, restriction fragment length polymorphism assays, single stranded conformational polymorphism analyses, in situ hybridization assays, and polymerase chain reaction analyses. Such analyses may reveal both quantitative aspects of the expression pattern of the gene, and qualitative aspects of the gene expression and/or gene composition. That is, such aspects may include, for example, point mutations, insertions, deletions, chromosomal rearrangements, and/or activation or inactivation of gene expression.

Preferred diagnostic methods for the detection of gene-specific nucleic acid molecules may involve for example, contacting and incubating nucleic acids, derived from the cell type or tissue being analyzed, with one or more labeled nucleic acid reagents under conditions favorable for the specific annealing of these reagents to their complementary sequences within the nucleic acid molecule of interest. Preferably, the lengths of these nucleic acid reagents are at least 9 to 30 nucleotides. After incubation, all non-annealed nucleic acids are removed from the nucleic acid:fingerprint molecule hybrid. The presence of nucleic acids from the fingerprint tissue that have hybridized, if any such molecules exist, is then detected. Using such a detection scheme, the nucleic acid from the tissue or cell type of interest may be immobilized, for example, to a solid support such as a membrane, or a plastic surface such as that on a microtitre plate or polystyrene beads. In this case, after incubation, non-annealed, labeled nucleic acid reagents are easily removed. Detection of the remaining, annealed, labeled nucleic acid reagents is accomplished using standard techniques well-known to those in the art.

Alternative diagnostic methods for the detection of gene-specific nucleic acid molecules may involve their amplification, e.g., by PCR (the experimental embodiment set forth in Mullis U.S. Pat. No. 4,683,202 (1987)), ligase chain reaction (Barany, Proc. Natl. Acad. Sci. USA, 88:189-93 (1991)), self sustained sequence replication (Guatelli, et al., Proc. Natl. Acad. Sci. USA, 87:1874-78 (1990)), transcriptional amplification system (Kwoh, et al., Proc. Natl. Acad. Sci. USA, 86:1173-77 (1989)), Q-β Replicase (Lizardi et al., Bio/Technology, 6:1197 (1988)), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment of such a detection scheme, a cDNA molecule is obtained from an RNA molecule of interest (e.g., by reverse transcription of the RNA molecule into cDNA). Cell types or tissues from which such RNA may be isolated include any tissue in which wild type fingerprint gene is known to be expressed, including, but not limited to, platelets, neutrophils and lymphocytes. A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method may be chosen from among the gene nucleic acid reagents described herein. The preferred lengths of such nucleic acid reagents are at least 15-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

Antibodies directed against wild type or mutant gene peptides may also be used as DMD diagnostics and prognostics. Such diagnostic methods, may be used to detect abnormalities in the level of gene protein expression, or abnormalities in the structure and/or tissue, cellular, or subcellular location of fingerprint gene protein. Structural differences may include, for example, differences in the size, electronegativity, or antigenicity of the mutant fingerprint gene protein relative to the normal fingerprint gene protein.

Protein from the tissue or cell type to be analyzed may easily be detected or isolated using techniques that are well known to those of skill in the art, including, but not limited to, western blot analysis. The protein detection and isolation methods employed herein may also be such as those described in Harlow and Lane, for example, (Antibodies: A Laboratory Manual, Cold Spring Harbor Laboratory Press, Cold Spring Harbor, N.Y. (1988)).

Preferred diagnostic methods for the detection of wild type or mutant gene peptide molecules may involve, for example, immunoassays wherein fingerprint gene peptides are detected by their interaction with an anti-fingerprint gene-specific peptide antibody.

For example, antibodies, or fragments of antibodies useful in the present invention may be used to quantitatively or qualitatively detect the presence of wild type or mutant gene peptides. This can be accomplished, for example, by immunofluorescence techniques employing a fluorescently labeled antibody (see below) coupled with light microscopic, flow cytometric, or fluorimetric detection. Such techniques are especially preferred if the fingerprint gene peptides are expressed on the cell surface.

The antibodies (or fragments thereof) useful in the present invention may, additionally, be employed histologically, as in immunofluorescence or immunoelectron microscopy, for in situ detection of fingerprint gene peptides. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the fingerprint gene peptides, but also their distribution in the examined tissue. Using the present invention, those of ordinary skill will readily perceive that any of a wide variety of histological methods (such as staining procedures) can be modified in order to achieve such in situ detection.

Immunoassays for wild type, mutant, or expanded fingerprint gene peptides typically comprise incubating a biological sample, such as a biological fluid, a tissue extract, freshly harvested cells, or cells that have been incubated in tissue culture, in the presence of a detectably labeled antibody capable of identifying fingerprint gene peptides, and detecting the bound antibody by any of a number of techniques well known in the art.

The biological sample may be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled gene-specific antibody. The solid phase support may then be washed with the buffer a second time to remove unbound antibody. The amount of bound label on solid support may then be detected by conventional means.

The terms “solid phase support or carrier” are intended to encompass any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet or test strip. Preferred supports include polystyrene beads. Those skilled in the art will know many other suitable carriers for binding antibody or antigen, or will be able to ascertain the same by use of routine experimentation.

The binding activity of a given lot of anti-wild type or -mutant fingerprint gene peptide antibody may be determined according to well known methods. Those skilled in the art will be able to determine operative and optimal assay conditions for each determination by employing routine experimentation.

One of the ways in which the gene peptide-specific antibody can be detectably labeled is by linking the same to an enzyme and using it in an enzyme immunoassay (EIA) (Voller, Ric Clin Lab, 8:289-98 (1978) “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol., 31:507-20 (1978); Butler, Meth. Enzymol., 73:482-523 (1981); Maggio (ed.), Enzyme Immunoassay, CRC Press, Boca Raton, Fla. (1980); Ishikawa, et al., (eds.) Enzyme Immunoassay, Igaku-Shoin, Tokyo (1981)). The enzyme that is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type, mutant, or expanded peptides through the use of a radioimmunoassay (RIA) (See, e.g., Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediamine-tetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Alternative diagnostic and prognostic methods for the detection of muscular dystrophy are provided in the form of blood testing for the presence and function of a GAMT enzyme. The GAMT enzyme is normally present in the blood of a mammal in very low or undetectable levels. However, in the blood of a mammal with a muscular dystrophy, the GAMT enzyme may be found at moderate to elevated levels. Similar to creatine and creatine kinase, intracellular GAMT enzyme leaks from the cells of muscular dystrophy patients to enter the blood stream where it is found in detectable levels. For this reason, the blood of a mammal having, or suspected of having, a muscular dystrophy may be tested for the presence of the GAMT enzyme as a marker indicative of a muscular dystrophy in the mammal. The presence of elevated serum GAMT enzyme is itself an important marker, indicative of a mammal suffering a muscular dystrophy. Thus, in a young male (less than 5 years of age) with suspected of having DMD, the ratio of higher levels of GAMT/elevated serum CK in the blood of a mammal could predict a less severe form of muscular dystrophy. In another young male suspected of having DMD, the ratio of lower GAMT/highly elevated serum CK could predict a more severe form of muscular dystrophy. The GAMT:CK ratio may be an important screening tool for stratifying modifier factors of a individual's disease risk and prognosticating the clinical course of a boy with muscular dystrophy. This approach could also be used in other forms of muscular dystrophy in addition to DMD. The current screening test of serum CK indicates a patient with likely dystrophinopathy but does not offer additional insight of who could be in the spectrum of benign or deleterious forms of muscular dystrophy e.g. Beckers vs Duchenne muscular dystrophies. Specific DNA testing of dystrophin genes for in-frame or out-of-frame mutations, duplications, deletions or point mutations are also unable to fully predict the phenotypic severity of some dystrophinopathy patients. This diagnostic method may be extended further to test the enzymatic activity of a GAMT enzyme found in the blood of a mammal. This enzymatic activity assay provides additional evidence about the mammal's ability to produce creatine. For example, high levels of a mutated or otherwise dysfunctional GAMT enzyme in the blood of a mammal would be prognostic of a more severe form of a muscular dystrophy exacerbated by a dysfunctional GAMT enzyme in the mammal, and therefore a limited ability to form endogenous creatine. Alternatively, a low level of a functional GAMT enzyme in the blood of a mammal would be prognostic of a less severe form of a muscular dystrophy in a patient likely to be capable of forming endogenous creatine. Thus, one embodiment of the present invention is a diagnostic test for the presence of a GAMT enzyme in the blood of a mammal having, or suspected of having, a muscular dystrophy. This embodiment may further comprise an evaluation of the enzymatic activity of any GAMT enzyme found in the blood of the mammal. Preferably, this diagnostic test is performed by exposing the mammal's blood, or some fraction thereof, to an antibody capable of recognizing a GAMT enzyme. The enzymatic activity of a GAMT enzyme appearing in the blood of a mammal is preferably tested by a spectrophotometric or fluorimetric assay.

The foregoing description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A transgenic mammal comprising a disruption in a guanidinoacetate methyltransferase (GAMT) gene and a dystrophin gene, wherein when the disruption is homozygous, the transgenic mammal lacks production of functional guanidinoacetate methyltransferase protein.

2. The transgenic mammal of claim 1, further comprising a disruption in an arginine:glycine amidinotransferase (AGAT) gene, wherein when the disruption is homozygous, the transgenic mammal lacks production of functional arginine:glycine amidinotransferase protein.

3. A cell isolated from the transgenic mammal of claim 1.

4. A method of identifying an agent effective for the prevention, treatment or amelioration of symptoms of a muscular dystrophy comprising:
administering an effective amount of a putative therapeutic agent to a transgenic animal of claim 1;
comparing the response of the transgenic animal to a control animal, wherein a response by the transgenic animal indicative of overcoming or lessening in the symptoms of a muscular dystrophy is indicative of effective treatment of a muscular dystrophy by the agent.

5. A method of treating a muscular dystrophy comprising modulating a gene selected from the group consisting of guanidinoacetate methyltransferase, arginine:glycine amidinotransferase, and a creatine transporter gene in a mammal.

6. The method of claim 5, wherein the modulating comprises administering to the mammal an agent that increases the expression of the gene.

7. The method of claim 5, wherein the modulating comprises administering to the mammal an agent that increases the activity of the gene product.

8. The method of claim 5, wherein the modulating comprises administering to the mammal a construct comprising the gene wherein the gene is expressed to produce the gene product in the mammal.

9. A diagnostic test for a muscular dystrophy comprising testing the blood of a mammal suspected of having a muscular dystrophy for the presence of a GAMT enzyme.

10. The diagnostic test of claim 9, further comprising testing of the enzymatic activity of a GAMT enzyme found in the blood of the mammal.
